SEQUENCE LISTING (2 sequences)

SEQ ID NO: 1 - DNA, 25 bp, Artificial Sequence
  Description of Artificial Sequencesynthetic DNA probe
atcacgcgaggtcttgcgatccccc

SEQ ID NO: 2 - DNA, 50 bp, Artificial Sequence
  Description of Artificial Sequencesynthetic DNA probe
atcggccgctccaatcacgcgaggtcttgcgatcccccgcttaccccctc

(19) United States
(12) Patent Application Publication
(10) Pub. No.: US 2004/0121339 A1
(43) Pub. Date: Jun. 24, 2004

(54) Special film-coated substrate for bio-microarray fabrication and use thereof

(76) Inventors: Jizhong Zhou, Oak Ridge, TN (US); Xichun Zhou, Oak Ridge, TN (US)

Correspondence Address: QUARLES & BRADY LLP, 411 E. WISCONSIN AVENUE, SUITE 2040, MILWAUKEE, WI, 53202-4497, US

(21) Appl. No.: 10/326,031

(22) Filed: Dec. 19, 2002

(51) Int. Cl. C12Q 1/68; C12M 1/34

(52) U.S. Cl. 435/6; 435/287.2; 435/7.1

(57) ABSTRACT

Platforms for easy and cost-effective fabrication of bio-microarrays are disclosed. In one embodiment, the platform contains a substrate having a surface coated with a film of alternating polycationic and polyanionic polymers. In another embodiment, the platform contains a substrate having a surface coated with a polyelectrolyte-silica composite film. Also disclosed are bio-microarrays fabricated using the above platforms and methods of making the platforms and the microarrays.

### CROSS-REFERENCE TO RELATED APPLICATIONS

Not Applicable

### STATEMENT REGARDING FEDERALLY SPONSORED RESEARCH OR DEVELOPMENT

This invention was made with United States government support awarded by the following agency: Department of Energy, Grant Number KP1301010. The United States has certain rights in this invention.

### BACKGROUND OF THE INVENTION

DNA and protein microarray technology is a rapidly growing technology and has impacted the genomic and proteomic research and commerce tremendously (1, 2). Microarrays utilize high-speed precision robots to affix thousands of biological samples onto a solid support (a glass, silica or nylon membrane slide). Through spatial multiplexing, thousands of genes or proteins can be evaluated simultaneously using microliters of sample. This has allowed for the collection of massive amounts of data in a short period of time and has proven to be an important milestone in biotechnology. Microarrays are also important tools for drug discovery, diagnostics and toxicology studies.

One crucial component of the microarray technology is the platform substrate that is suitable for spotting and then immobilizing a variety of biological active molecules including DNA, proteins and cells for further biomolecular interaction evaluation. Currently, two predominant methods are used to produce oligonucleotide microarrays. One is to synthesize oligonucleotides in-situ on a substrate by using photo protection groups and masks to direct the selective addition of nucleotides (developed by Affymetrix Inc.) (3, 4). The other is to synthesize oligonucleotide probes ex-situ, followed by covalent attachment to a monolayer molecule (coating thickness less than 10 nm) derivatized substrate surface through a primary amine, thiol, or disulfide (5, 6, 7), homo- or hetero-functional linkers (8), or by use of silanized oligonucleotide probes (9). Such covalent coupling requires activation of the underlying planar surface with cross-linking reagents and/or modification of DNA molecules with a reactive group. While effective, surface derivatization with silanes and the use of cross-linker reagents involve the use, hazard, and expense of several toxic compounds. Probe modifications also add considerable expense to the microarray, especially for individual researchers who usually print a relatively low volume of a particular microarray but need to print many different kinds of microarrays for multiple projects.

Moreover, because of the planar surface of the slides used in the two predominant methods of producing microarrays, the capacity for the covalent immobilization is limited, resulting in a relatively low assay sensitivity. To overcome this problem, another approach by depositing a thick coating film with film thickness from 100 μm to 1 mm has been used to increase the number of probes that can be bound to the glass surface. Acrylamide gel pads (10, 11) or gelatin pads (12), structured by photolithography, as well as dendrimeric linker systems (13) that multiply the coupling sites by introducing additional reactive groups through branched linker molecules are some examples of this attempt to enhance the performance of miniaturized glass slide-based hybridization studies. All approaches attempt to combine the properties of the glass support, simple handling and detection with the binding capacity of filter membranes. Most procedures require several synthesis steps and in some cases include photolithographic activation (10-12). The synthesis procedures are time-consuming and expensive and will change the physical properties of the slide, which will result in high background signal or inaccessibility of the attached probes. In addition, skilled workers are required to conduct the chemical synthesis steps and photolithographic activation. This is not practical for individual researchers and laboratories.

Protein miroarrays are useful for a variety of applications, such as identifying protein-protein interactions, enzyme assays, drug screening, tissue and serum protein profiling, and antibody characterization. However, protein-based microarrays face several additional challenges. Proteins are generally attached and analyzed on activated aldehyde slides (14), where the primary amines and amino terminal amines of the proteins can react readily with the aldehydes of the slide to form a covalent bond. However, in general, proteins are more sensitive to their surrounding environment than are nucleic acids. The hydrophobic nature of many glass and plastic surfaces can cause protein denaturation. Thus, substrate choice is a major consideration when designing protein microarray experiments. Ideally, proteins should be immobilized on a slide support in a way that preserves their native format and their folded conformations. To increase binding capacity, porous substrates such as organic hydrogel nitrocellulose film have been used for fabricating protein microarrays. Protein microarrays produced on these slides suffer from high background signal and high cost because special equipment and engineering processes are required to produce an even film of hydrogel and nitrocellulose on slide surfaces. A microarray platform that is inexpensive and can be flexibly designed to suit special needs is in great demand for the fabrication of robust and high-throughput protein microarrays.

### SUMMARY OF THE INVENTION

In one aspect, the present invention relates to a platform for fabricating bio-microarrays wherein the film contains a solid substrate and a special film coating on a surface of the substrate. In one embodiment, the special film has a structure of alternating layers of polycationic and polyanionic polymers. In another embodiment, the special film is a polyelectrolyte-silica composite film. These films are rich in electric charges, three dimensional porous structures and hydrogen bond forming groups and thus can immobilize biological matters by electrostatic and porous adsorptions and potentially hydrogen bonds as well.

In another aspect, the present invention relates to a bio-microarray that contains a biological matter immobilized onto the film of a platform described above.

In still another aspect, the present invention relates to a method of making a platform for fabricating bio-microarrays. The method involves providing a substrate suitable for fabricating bio-microarrays and coating a surface of the substrate with a special film described above.

In yet another aspect, the present invention relates to a method of making bio-microarrays. The method involves providing a platform as described above and attaching a biological matter to the film of the platform.

It is an object of the present invention to provide a platform for easy and cost-efficient fabrication of bio-microarrays.

It is a feature of the present invention that no modifications to biomolecules are required prior to their immobilization onto the platform for forming a microarray. Accordingly, the biomolecules on the microarray can be kept in their native form and are freely accessible for binding.

It is another feature of the present invention that no post-array chemical reactions such as blocking residue reaction sites on a platform is required.

It is an advantage of the present invention that the platforms provided for fabricating bio-microarrays are compatible with most commercial printing (spotting) technologies and scanning analysis equipments.

Other advantages, features and objects of the invention will become apparent from the following detailed description when considered in conjunction with the accompanying claims and drawings.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All references mentioned herein are incorporated by reference in their entirety. In case of conflict, the present specification, including definitions, will control.

### BRIEF DESCRIPTION OF THE DRAWINGS

FIG. 1 shows embodiments of procedures for preparing special film-coated slides and microarrays.

FIG. 2 shows oligonucleotide microarray images of special film-coated slides and commercial slides.

FIG. 3 shows hybridization signal intensity of special film-coated slides and commercial slides.

FIG. 4 compares the average background signal and spot size on special film-coated slides and commercial slides.

FIG. 5 shows the discrimination factor (which is defined as the ratio of fluorescent intensity of mismatched probes to that of the perfect match) on special film-coated slides and commercial slides.

FIG. 6 shows detection of protein-protein interaction on special film-coated glass slides. Slide probed with: 1, mixed Human IgG-Cy3, Fibronectin-Cy3, and Biotin-BSA Cy3; 2, Human IgG-Cy3 and Biotin-BSA Cy3; 3, Human IgG-Cy3; 4, Fibronectin-Cy3; 5, Biotin-BSA Cy3. BSA was used as a negative control on the microarrays.

FIG. 7 shows the dynamic range of a protein microarray.

FIG. 8 shows images of protein arrays on special film-coated slides and two kinds of commercially available slides.

### DETAILED DESCRIPTION OF THE INVENTION

The term bio-microarray is used in the specification and claims to mean a microarray of biological matters. Examples of biological matters that can form a microarray include biomolecules such as polynucleotides and polypeptides, prokaryotic or eukaryotic cells, organelles of prokaryotic or eukaryotic cells, and plant or animal tissue samples.

The term polynucleotide is used in the specification and claims to mean a molecule that contains a sequence of ribonucleotides or deoxyribonucleotides. Thus, the term covers both DNA and RNA molecules. The sequence of deoxyribonucleotides or ribonucleotides can be short (e.g., oligonucleotide) or long (e.g., PCR amplicon, genomic DNA). In addition to the deoxyribonucleotides or ribonucleotides, a polynucleotide as defined herein may also contain chemically or enzymatically modified nucleotides such as nucleotide analogs.

The term polypeptide is used in the specification and claims to mean a molecule that contains an amino acid sequence. The amino acid sequence can be short (e.g., a short peptide) or long (e.g., a full length protein). A polypeptide as defined herein may also contain chemically or enzymatically modified amino acids.

The term polyelectrolyte, polyionene and polyionic polymer are used synonymously in the specification and claims to mean a polymer that has multiple ionic or ionizable groups of the same charge. Similarly, the term polycationic or polyanionic polymer is used to refer to a polyionic polymer with the positive or negative charge.

In order to provide a platform that can be used for fabricating polynucleotide or polypeptide microarrays without having to modify the biomolecules during the fabrication process, the inventors have developed a solid microarray substrate coated with a special film that is rich in electric charges, three dimensional porous structures and hydrogen bond forming groups. In one embodiment, the special film has a structure of alternating layers of polycationic and polyanionic polymers (referred to as the polyelectrolyte film or three-dimensional polyelectrolyte film). In another embodiment, the special film is a polyelectrolyte-silica composite film (also referred to as the three-dimensional polyelectrolyte-silica composite film), which is film that contains silica and a polyelectrolyte. Polynucleotides, polypeptides and other biological matters (e.g., cellular organelles, cells and tissue samples) can be immobilized onto these films by electrostatic and porous adsorption and potentially hydrogen bond as well. As shown in the examples below, the polyelectrolyte and the polyelectrolyte-silica composite films have a low fluorescence background and thus can be used in hybridization and binding assays using fluorescence as the detection method.

Any substrates that are known to a skilled artisan as suitable for fabricating bio-microarrays can be used in the platform of present invention. Typically, the solid substrates or the coated surface of the solid substrates are planar in shape. Examples of suitable substrates include but are not limited to those that are made of glass, silica or plastic (e.g., nylon). Preferably, the thickness of a polyelectrolyte film ranges from 1 nanometer to 100 micrometers, from 5 nanometers to 20 micrometers, or from 50 nanometers to I micrometer; the thickness of a polyelectrolyte-silica composite film ranges from 1 micrometer to 10 millimeters, from 10 micrometer to 2 millimeters, or from 100 micrometers to 1 millimeter.

There are many ways that a film of alternating layers of polyionic polymers of opposite charges can be formed and the present invention is not limited to any particular way of forming the film. For example, polyionic polymers containing a desirable number of ion groups can be deposited onto a solid substrate directly. Alternatively, a polymer of no or lower than desirable number of ion groups can be deposited and additional ion groups can then be introduced into the polymer layer. A skilled artisan is familiar with the techniques for depositing a polymer onto a solid substrate (see e.g., references 15 and 16, which are herein incorporated by reference in their entirety). In a preferred embodiment of the present invention, each layer of the film is formed by self-assembly process and the multi-layers are stacked together by the electrostatic attraction between oppositely charged polyelectrolytes (for self-assembly process, see e.g., reference 17, which is herein incorporated by reference in its entirety). In this embodiment, a solid substrate is exposed to a solution of a first polyionic polymer allowing the formation of a layer of the polymer through self-assembly process. Next, the substrate covered with the first polymer is exposed to a solution of a second polyionic polymer that is of the opposite charge of the first polymer to allow formation of a layer of the second polymer on top of the layer of the first polymer. This process is repeated with additional polyionic polymers until a desired number of layers are reached. This method has been shown to produce films of high uniformity (surface roughness <10 Å) and of a well-defined (controllable) thickness. The films created with this technique provide a biological friendly, solution-like environment for biological immobilization arid are well suited for microarray fabrication. In this preferred embodiment, the film can be formed without any synthesis work and special equipment.

Each polyionic polymer used for forming the polyelectrolyte film in the present invention has multiple ionic or ionizable functional groups of the same charge. Specifically, the functional groups of the same charge are cations, groups that can be ionized to cations, anions, or groups that can be ionized to anions. Different cations or anions or groups that can be ionized thereto can be represented in a particular polymer. However, for reasons of accessibility and ease of production, it is preferable that multiple ionic groups in a polymer be identical. Preferably, the polymers used in the present invention are soluble. More preferably, the polymers are soluble in an aqueous solution. Examples of aqueous soluble polymers that can be used in the present invention include but are not limited to polyallylamine hydrochloride (PAAH), polyethyleneimine (PEI), polydimethyldiallylammonium chloride (PDDA), polyacrylamide hydrochloride (PAAM), poly(4-vinylpyridine) hydrochloride (PVP), polystyrenesulfonate (PSS), polyvinylsulfonate (PVS), dextrinsulfate (DTS), and analogs of the foregoing polymers. The structures of PAAH, PEI, PDDA, PAAM, PVP, PSS, PVS and DTS are illustrated below:

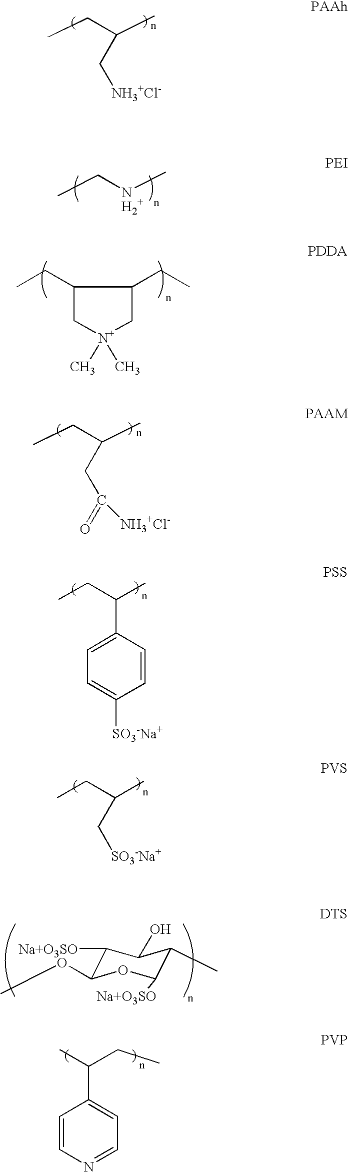

To form a polyelectrolyte film on a solid substrate, a suitable solvent must be used to dissolve the polymers in a homogenous solution. Although water is of somewhat an advantage as it can dissolve the polyelectrolyte alone, the solvent used can vary, depending somewhat upon the polymer being adsorbed. Mixed water-miscible solvents, e.g., water-acetone, water-ethanol and water-tetrahydrofuran (THF), can also be used. The optimum concentration of the polymer can be readily determined by those skilled in the art.

One or more salts can also be added into a polyelectrolyte solution to increase the roughness of the film and thereby facilitating immobilization of a greater amount of biological matters onto the film. Preferably, the salts are inorganic salts. Examples of salts that can be added include but are not limited to salts of Mn²⁺, Cu²⁺, Fe²⁺, Na⁺, NH₄⁺, K⁺, Ni²⁺ and Mg²⁺.

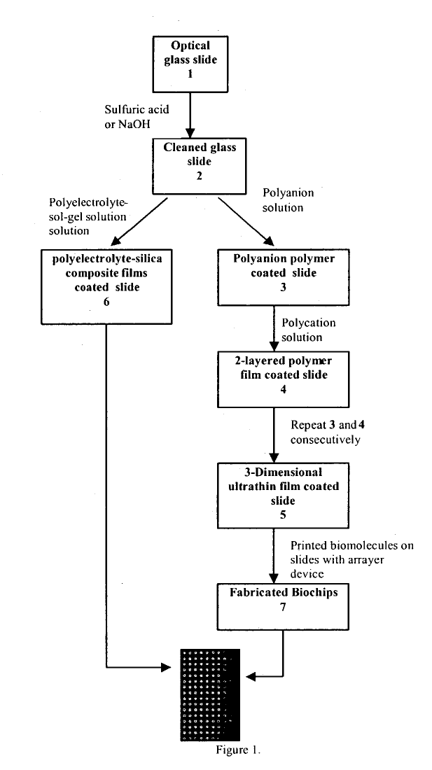

Multi-layer films used in the present invention contain at least two polymers that have ionic groups of opposite charges. Thus, the simplest layer sequence is of the ABABAB . . . type in which A represents one layer and B represents another. However, the functionality of the layers can be selectively increased by using more than two polymers, for example, ABCBABABCB . . . or ABCDCBADCBAD . . . , in which A and C carry the same charge and B and D carry the same charge opposite to that of A and C. The layer sequence is a consequence of the order of exposure used to apply the individual layers. Slides are preferably rinsed between individual applications to remove residual amounts of polymers that have not bonded or have been only loosely adsorbed to the support. The process for applying the layers of film can easily be converted into a continuous procedure by alternately passing the modified substrate through baths containing the polymers with solvents, and baths containing rinsing liquids. FIG. 1 illustrates the procedures for preparing the film-coated slide and the micro arrays.

An example protocol of producing a platform containing a polyelectrolyte film and a microarray on the platform is as follows (FIG. 1): (1) Optical glass slide 1 is cleaned, then the cleaned glass slide 2 is dipped into a polyanionic polymer solution to deposit a polymer layer 3 on the surface, and then dipped into a polycationic polymer solution to deposit a polycationic polymer layer 4. The 3-dimensional polyelectrolyte film coated slide 5 is created by repeating five or six alternate adsorptions of every type of polyelectrolyte pair such as PSS/PAAH, PSS/PEI, PVS/PEI, DTS/PAAH, PVS/PDDA, PSS/PDDA, and DTS/PDDA. Sodium polystyrenesulfonate (PSS, MW 70,000, Aldrich Co.) at a concentration of 3 mg/ml, polyallylaminehydrochloride (PAAH, MW 50,000-65,000, Aldrich Co.), polyvinylsulfonate (PVS), polydimethyldiallylammonium chloride (PDDA, Aldrich Co.) at a concentration of 2 mg/mL, dextrinsulfate (DTS) at a concentration of 1.5 mg/mL and branched polyethyleneimine (PEI, MW 70,000) at a concentration of 1.5 mg/mL are dissolved in pure water. The pHs of the solutions are adjusted by adding HCl or NaOH. The outermost layer of the slide becomes “negative” or “positive,” accordingly. Then the coated glass slide can be used to fabricate the polynucleotide or polypeptide microarray 7. In polynucleotide microarrays, the outermost layer of the film is usually positive; in polypeptide microarrays, the outermost layer of the film is usually negative.

There are many ways that a solid substrate suitable for fabricating microarrays can be coated with a polyelectrolyte-silica composite film and the present invention is not limited by any particular way that the substrate is coated with the film. Generally speaking, the film-coated substrate can be made by adding a polyelectrolyte into a silica sol-gel solution and dipping a solid substrate into the solution. Alternatively, a spin coating method (18) can be used to deposit the polyelectrolyte doped sol-gel solution onto a surface of a solid substrate (see FIG. 1). A skilled artisan is familiar with the sol-gel techniques and the coating techniques that can be used to coat a substrate with a polyelectrolyte-silica composite film.

The polyelectrolytes that can be used to form a polyelectrolyte-silica composite film are as defined and described above for the polyelectrolyte film. Typically, a polycationic polymer is used for a platform for fabricating a polynucleotide microarray and a polyanionic polymer is used for a platform for fabricating a polypeptide microarray. The polyelectrolyte-silica composite film can be formed with any silica sol-gel material. Examples of the sol-gel materials include but are not limited to those made of aminoalkylsiloxanes, aminocarboxyalkylsiloxanes, carboxyalkylsiloxanes, alkoxaysilanes, and a combination thereof.

The invention will be more fully understood upon consideration of the following non-limiting examples.

### EXAMPLE 1

Fabrication of Oligonucleotide Microarray on Polyelectrolyte Film Coated Glass Slides

This example describes one way to fabricate oligonucleotide microarrays by self-assembly immobilization of 20-mer length probes onto polyelectrolyte films. The optical glass slide was cleaned with hot Piranha solution (30% H₂O₂:H₂SO₄/1:3), and then thoroughly rinsed with distilled water and HPLC purified ethanol. The surface was blown dry with nitrogen steam or in dust-free ambient air. The cleaned slide substrate was first immersed into a 1 mM aminopropyltrimethoxylsilane/ethanol solution for 30 minutes to form a self-assembled monolayer film on the glass surface (amino groups towards the outside), which was then able to interact with the polyelectrolytes under certain conditions. Polyelectrolyte adsorption was then performed as follows. The slide substrate was immersed in 50 mL of 3 mg/mL PSS aqueous solution with a pH value of approximately 2 for 5 minutes, followed by washing with water, and drying in nitrogen or dust-free ambient air. The PSS-coated slide was then exposed to 5 mL of 3 mg/mL PAAH solution (pH 8.0, adjusted by adding NaOH) for 5 minutes. This surface was then washed with pure water and dried with nitrogen or air. This procedure was repeated until 10 polyelectrolyte layers (PSS/PAAH)₅ were deposited.

Oligodeoxyribonucleotide printing solutions were prepared in a dilution series from 50 μM to 0.09765 μM in 50% DMSO (Sigma, St. Louis, Mo.)/50% H₂O. Ten microliters of each printing solution was transferred to a 384-well source plate for printing. DNA samples were arrayed with a single pin at a spacing distance of 250 μM in 144, 16×9 patches on the above polyelectrolyte coated slide by using a PixSys 5500 robotic printer (Cartesian Technologies, Inc., Irvine, Calif.) in 60% relative humidity. After printing, the DNA was fixed to the slides by UV cross-linking at 65 mJ in a UV Stratalinker 1800 (Stratagene, La Jolla, Calif.). The glass slides were then rinsed with deionized water and dried in air or are centrifuged at 500 g for 5 minutes.

The oligonucleotide used in this study has the following sequence: 5′-ATCACGCGAGGTCTTGCGATCCCCC-3′ (SEQ ID NO:1).

Before hybridization, the fabricated oligonucleotide microarrays were prehybridized with 0.5% BSA, 0.1% SDS in 100 mM PBS buffer for 10 minutes. The prehybridization buffer was removed by centrifugation at 500 g for 5 minutes. Cy3-labelled complementary oligonucleotides were dissolved in 50% 3×SSC, 50% Formamide, and 0.2% SDS hybridization buffer (1×SSC contained 150 mM NaCl and 15 mM trisodium citrate) at a concentration of 0.1 μg/mL. Ten microliters of hybridization fluid was applied to the DNA microarray and a glass coverslip (6.25×8 mm) was then applied to the slide. Hybridization was carried out for 6-14 hrs at 50° C. Following hybridization, the arrays were washed with 1×SSC, 0.2% SDS, 0.1×SSC, and 0.2% SDS for 5 minutes each at ambient temperature and then with 0.1×SSC for 30 seconds also at ambient temperature prior to being dried in air or by centrifugation at 500 g for 5 minutes. The microarrays were scanned with the scanning laser confocal fluorescence microscope of the ScanArray 5000 System.

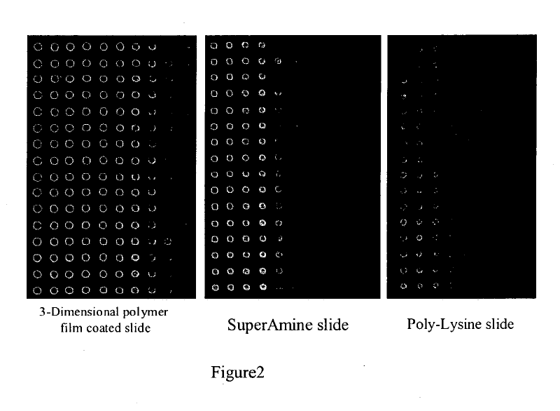

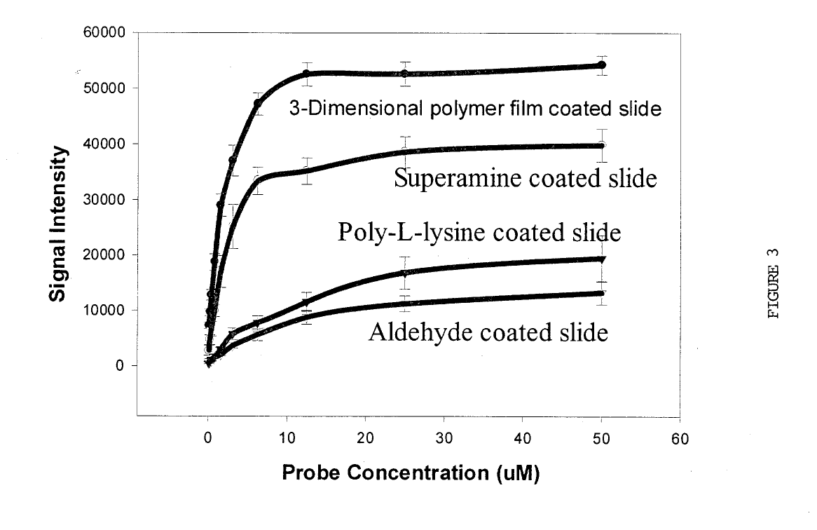

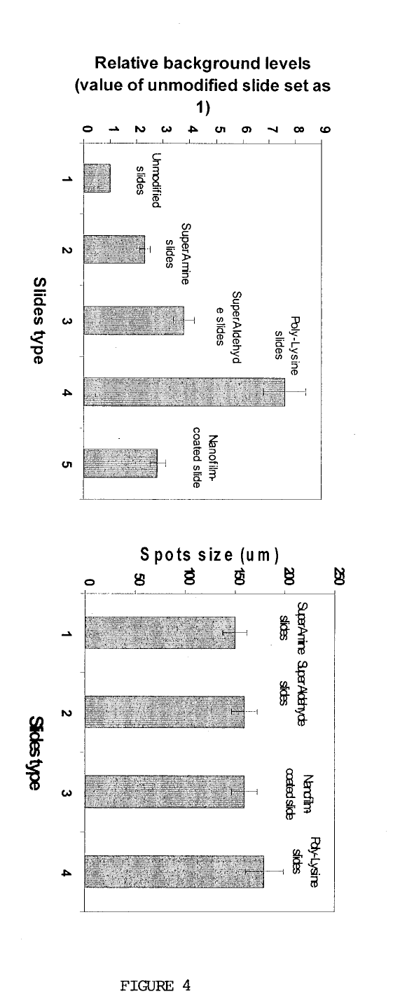

For comparison, oligonucleotide microarrays were also fabricated on three types of commercially available glass slides under identical conditions, and hybridizations were also carried out under identical conditions. FIG. 2 shows images of the oligonucleotide microarrays of the 3-dimensional polyelectrolyte film coated slides as compared to the commercial slides. Microarrays on the 3-dimensional polyelectrolyte film coated slides had consistent spot morphology. FIG. 3 shows the comparison of the hybridization signal intensity of the 3-dimensional polyelectrolyte film coated slides and the commercial slides. The signal intensity of the 3-dimensional polyelectrolyte film coated slides was at least 1.5 times higher than the commercialized SuperAmine glass slide, which indicates the presence of higher binding efficiency and better hybridization kinetics on the former slides. FIG. 4 compares the average background signal and spot size of 144 spots from the 3-dimensional polyelectrolyte film coated slides to those of the commercially available slides. It shows that the background signal of the 3-dimensional polyelectrolyte film coated slides was lower than that of commercially available slides (readings for unmodified glass slides were assigned a value of 1, all other readings were normalized to this value). The spot sizes of microarrays for the 3-dimensional polyelectrolyte film coated slides were the same as those of the commercially available slides. Finally, the spots on the 3-dimensional polyelectrolyte film coated slides were more consistent (i.e., had lower variation).

### EXAMPLE 2

### Detection of Signal Nucleotide Mismatches on Polyelectrolyte Film Coated Glass Slides

This example describes an alternate method of fabricating oligonucleotide microarrays on 3-dimensional polyelectrolyte film coated slides and the use of the microarray to detect single nucleotide mismatches. The optical glass slide was cleaned with 2.5 M NaOH for 2 hours, thoroughly rinsed with distilled water. The cleaned slide substrate was then immersed in 50 mL of 3 mg/mL PEI solution (pH 8.0, adjusted by adding NaOH) for 5 minutes followed by washing with water and exposure to 50 ml of 1.5 mg/mL PSS aqueous solution with a pH value of approximately 2 for 5 minutes. This surface was then washed with pure water and dried with nitrogen or air. The whole procedure was repeated until 12 polyelectrolyte layers (PEI/PSS)₆ were deposited on the glass surface. Finally, the slide is immersed in 50 mL of 3 mg/mL PEI solution (pH 8.0, adjusted by adding NaOH) for 5 minutes to form the 3-dimensional polyelectrolyte film with the out layer positive charged. The polyelectrolyte film coated slides were then dried with nitrogen or dust-free ambient air.

To determine whether single mismatch discrimination can be achieved with microarray hybridization for 16S genes, a model oligonucleotide microarray derived from a region of 16S genes was designed and fabricated on the polyelectrolyte film coated slide. The 16S probes contained 1-5 mismatches at various positions. A target template, 50 bp in length and labeled with Cy3 fluorescent dye, was also synthesized (the target template has the sequence of 5′-ATCGGCCGCTCCAATCACGCGAGGTCTTGCGATCCCCCGCTTACCCCCTC-3′ (SEQ ID NO:2)). 16S probes with the same sequence but with modification of amine-hexane (aminoC6) at the 5′-end, were also synthesized and printed on commercially available SuperAldhyde slides according the manufacturer's protocol.

After prehybridization with prehybrdization buffer (0.5% BSA, 0.1% SDS, in 100 mM PBS; or 1×Dehardt's solution) for 10 minutes, the buffer was removed by spinning at 500 g for 2 minutes. Ten μL of Cy3-labeled 50-bp target solution (50% Formiamde, 3×SSC buffer, 0.2% SDS) was applied to the microarray. Hybridization was carried out under a supported coverslip at 55° C. for 12 hours. Arrays were washed with 1×SSC, 0.2% SDS, 0.1×SSC, and 0.2% SDS for 5 minutes each at ambient temperature and then with 0.1×SSC for 30 seconds, also at ambient temperature, prior to being dried in air or by centrifugation at 500 g for 5 minutes. Slides were then scanned on a ScanArray 5000 System to detect Cy5 fluorescence. Fluorescent intensity was analyzed using Array Vision 4.0.

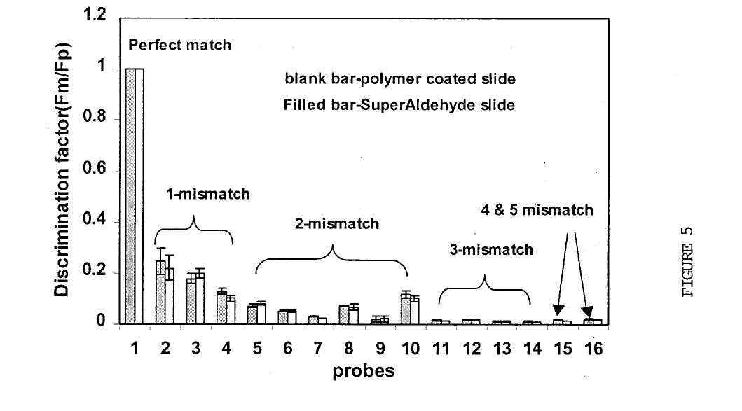

FIG. 5 shows the discrimination factor (which is defined as the ratio of fluorescent intensity of mismatched probes to that of the perfect match) on the 3-dimensional polyelectrolyte film coated slides and the commercial slides. The discrimination factor of the developed 3-dimensional polyelectrolyte film coated slides was the same level as that of the commercially available slides. This result indicates that the DNA arrays fabricated on the developed 3-dimensional polyelectrolyte film coated glass slides do not lose any specificity.

### EXAMPLE 3

### Protein Microarrays Fabricated on Polyelectrolyte Film Coated Glass Slides

This example describes a method of preparing 3-dimensional polyelectrolyte film coated glass slides and the fabrication of protein microarrays. The optical glass slide was cleaned with Piranha solution (30% H₂O₂:H₂SO₄/1:3), thoroughly rinsed with distilled water and HPLC purified ethanol, and then dried in air or in a dust-free oven at 50° C. The cleaned slide substrate was then immersed in 50 ml of 1.5 mg/mL PSS aqueous solution with a pH value of approximately 2 for 5 minutes, followed by washing with water and exposure to 50 mL of 3 mg/mL PAAH solution (pH 8.0, adjusted by adding NaOH) for 5 minutes. This surface was then washed with pure water and dried with nitrogen or air. The whole procedure was repeated until 12 polyelectrolyte layers (PSS/PEI)₆ were deposited on the glass surface. Finally, the glass slide was immersed in 50 ml of 1.5 mg/mL PSS aqueous solution with a pH value of approximately 2 for 5 minutes to form the 3-dimensional polyelectrolyte film with an outmost layer of negative charge. The polyelectrolyte film coated slides were then dried with nitrogen or dust-free ambient air.

Alternatively, the developed 3-dimensional polyelectrolyte film coated glass slide was prepared with the following method. Glass microscope slides were cleaned in 2.5 M NaOH for 2 hours, rinsed thoroughly in ultra-pure water, then soaked for 30 minutes in a 3 mg/mL PAAH solution (pH 8.0, adjusted by adding NaOH) for 5 minutes. They were then rinsed in ultra-pure H₂O, and then soaked in a 1.5 mg/mL PSS aqueous solution with a pH value of approximately 2 for 5 minutes. This procedure was repeated until 12 polyelectrolyte layers (PAAH/PSS)₆ were deposited on the glass surface. The slide was rinsed with ultra-pure water and spun dry.

Four antibody/antigen pairs were obtained from a commercial source (anti-human IgG and human IgG, anti-fibronectin and fibronectin, biotinlayted bovine serum albumin and streptavidin). Antibody probe printing solutions were prepared in a dilution series from 0.5 mg/mL to 0.0125 mg/mL in PBS (0.14 M NaCl, 0.003 M KCl, 0.01 M sodium phosphate) and source plates were set up in 384-well plates. The antibody probes were printed at a volume of 500 picoliters per spot, using an arrayer, on the prepared polyelectrolyte film coated glass slide. Following printing, the microarrays were incubated for 2 hours at 25° C. at 60% relative humidity. Slides were then washed three times for 5 minutes in a solution of PBS with 0.5% Tween 20 (PBST) to remove any unbound probes. Before immunoassay, the antibody arrays were blocked with 15 μL of 0.5% BSA, and 0.2% Tween 20 PBS solution for 15 minutes. The excess liquid was shaken off. Antibody microarray slides were stored in a solution of 0.5% BSA and 0.2% Tween 20 PBS solution at 4° C. Immunoassays were carried out with a Cy3-labeled antigen solution of 10 μg/mL in 100 mM PBS for 2 hours at room temperature. Without allowing the array to dry, 15 μL of dye-labeled antigen solution at 10 μg/mL in 100 mM PBS was applied to the microarray surface. A 24 mm×30 mm coverslip was placed over the solution. The arrays were sealed in a chamber with an under-layer of PBS to provide humidification, after which they were kept at room temperature for 2 hours. The arrays were dunked briefly in PBS to remove the protein solution and the coverslip, and they were allowed to rock gently in PBS/0.1% Tween 20 solution for 20 minutes. The arrays were then washed twice in PBS for 5-10 minutes each and twice in water for 2-5 minutes each. All washes were at room temperature. After spinning to dryness in a centrifuge, the arrays were scanned with a ScanArray 5000 System.

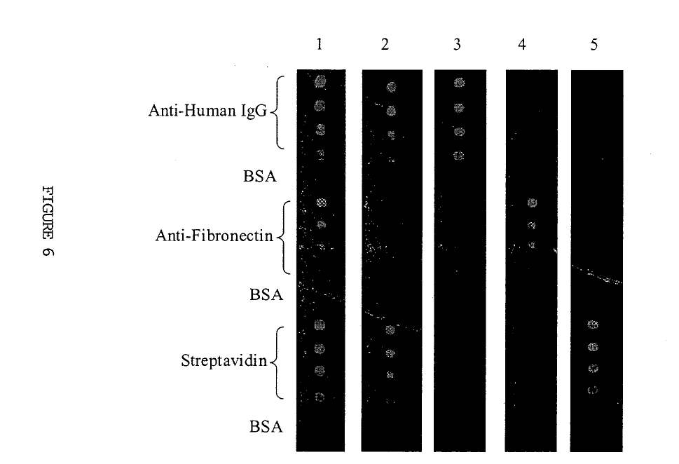

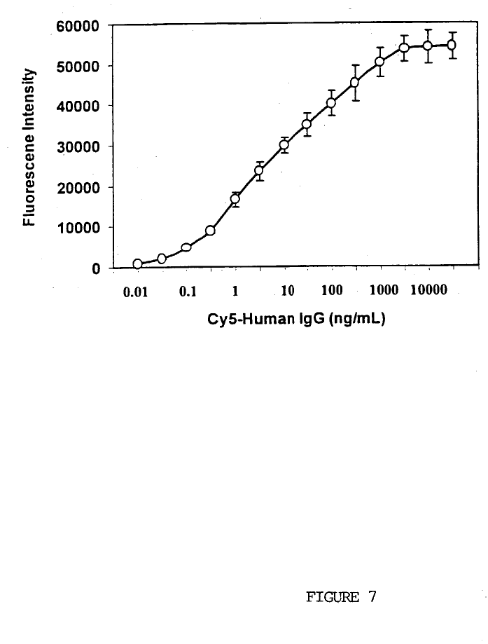

FIG. 6 shows the antibody-antigen interaction on the developed protein microarray. The detection was highly specific and no significant background or nonspecific immunoassaying occurred. To determine the range of sensitivity of this assay, we varied the concentration of both the protein being spotted (anti-human IgG) and the protein in solution (Cy3-human IgG). The signal of the spotted protein began to saturate at concentrations above 0.125 mg/mL. Below this, the fluorescent intensity scaled linearly with decreasing concentrations of anti-human IgG. In the case of solution-phase protein Cy3-human IgG, fluorescent intensity scaled linearly with protein concentration over four orders of magnitude (FIG. 7). Specific binding could be detected using Cy3-human IgG concentrations as low as 100 pg/mL.

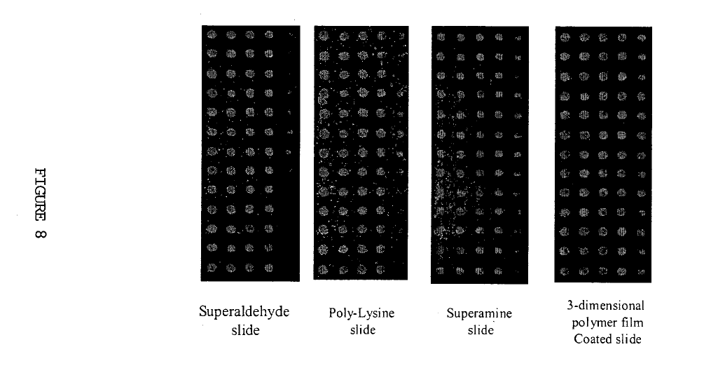

Antigen microarrays can also be fabricated on the 3-dimensional polyelectrolyte film coated slides. For example, biotin-conjugated BSA printing solution was prepared from concentrations of 0.125 mg/mL to 0.0039 mg/mL in PBS (0.14 M NaCl, 0.03 M KCl, 0.01 M sodium phosphate), and 10 μL of the each solution was transferred to 384-well plates. Protein samples were arrayed with a single pin at a spacing distance of 250 μm in 90, 16×5 patches on the above polyelectrolyte film coated slide by using a PixSys 5500 robotic printer (Cartesian Technologies, Inc., Irvine, Calif.) at 60% relative humidity. After printing, the microarrays were incubated for 2 hours at 25° C. in 60% relative humidity. Slides were then washed three times for 5 minutes in a solution of PBS with 0.5% Tween 20 (PBST) to remove any unbound probes. Before immunoassay, the antibody arrays were blocked with 15 μL of 0.5% BSA, 0.2% Tween 20 PBS solution for 15 minutes and the excess liquid was shaken off. Antibody microarrays were stored in a solution of 0.5% BSA, 0.2% Tween 20 PBS solution at 4° C. Immunoassays were carried out with Cy3-labeled, 101 μg/mL streptavidin solution in 100 mM PBS for 2 hours at room temperature. After washing, the slides were scanned using a fluorescence microscope. FIG. 8 shows the images of protein arrays on the 3-dimensional polyelectrolyte film coated slides and two kinds of commercially available slides. Microarrays on the 3-dimensional polyelectrolyte film coated slide had consistent spot morphologies and lower background signal when compared to commercially produced slides.

### EXAMPLE 4

### Polyelectrolyte-Silica Composite Film Coated Glass Slides for Fabrication of Protein Microarrays

This example describes a method of preparing 3-dimensional polyelectrolyte-silica composite film coated glass slides and the fabrication of protein microarrays. The optical glass slide was cleaned with 10 N NaOH solution, thoroughly rinsed with distilled water and HPLC purified ethanol, and then dried in air or in a dust-free oven at 50° C. Silica sol-gel stock solution was prepared by mixing 4.0 mL TEOS (tetraethxylorthosilicate), 2.0 mL of deionized water and 100 μL HCl. The sol-gel solution was stirred at room temperature for 3 h. The polyelectrolyte-silica composite cocktail solution was achieved by mechanically blending sol-gel stock solution with polystyrenesulfonate (PSS) aqueous solutions. The volume ratio of the appropriate polyelectrolyte solution to the silica sol-gel stock solution was chosen to control the composition of the composite film. The sol-gel derived films were prepared from the freshly formulated polyelectrolyte-containing sol-gel stock solutions by spin-coating the surface of glass slide. A typical procedure for the spin-coating of the films onto the glass slides was as follows: 200 μL of polyelectrolyte-containing sol-gel stock solutions was pipetted onto the surface of the glass slide which was then spun at 3,000 rpm for 30 seconds. The film was then dried under ambient room conditions overnight or longer. Alternatively, the polyelectrolyte-silica composite film can also be deposited on the glass slide surface by dipping the glass slide into the polyelectrolyte-containing sol-gel stock solutions. After washing with pure water and dried in air, the polyelectrolyte-silica composite film coated glass slides are ready for fabrication of protein microarray.

### EXAMPLE 5

### Polyelectrolyte-Silica Composite Film Coated Glass Slides for Fabrication of DNA Microarrays

This example describes a method of preparing 3-dimensional polyelectrolyte-silica composite film coated glass slides and the fabrication of DNA microarrays. The optical glass slide was cleaned with 10 N NaOH solution, thoroughly rinsed with distilled water and HPLC purified ethanol, and then dried in air or in a dust-free oven at 50° C. Silica sol-gel stock solution was prepared by mixing 4.0 mL MET (α-methyacryloxypropyltrimethoxysilane), 2.0 mL of deionized water and 100 μL HCl. The sol-gel solution was stirred at room temperature for 3 h. The polyelectrolyte-silica composite cocktail solution was achieved by mechanically blending sol-gel stock solution with polydimethylammonium chloride (PDDA) aqueous solutions. The volume ratio of the appropriate polyelectrolyte solution to the silica sol-gel stock solution was chosen to control the composition of the composite film. The sol-gel derived films were prepared from the freshly formulated polyelectrolyte-containing sol-gel stock solutions by spin-coating the surface of glass slide. A typical procedure for the spin-coating of the films onto the glass slides was as follows: 200 μL of polyelectrolyte-containing sol-gel stock solutions was pipetted onto the surface of the glass slide which was then spun at 3,000 rpm for 30 seconds. The film was then dried under ambient room conditions overnight or longer. Alternatively, the polyelectrolyte-silica composite film can also be deposited on the glass slide surface by dipping the glass slide into the polyelectrolyte-containing sol-gel stock solutions. After washing with pure water and dried in air, the polyelectrolyte-silica composite film coated glass slides are ready for fabrication of DNA microarray.

The present invention is not intended to be limited to the foregoing examples, but encompasses all such modifications and variations as come within the scope of the appended claims.

### REFERENCES

1. Lockhart, D. J., Winzeler, E. A. (2000) Genomics, gene expression and DNA arrays. Nature, 405, 827-836.

2. Lander, E. S. (1999) Arrays of hope. Nat. Genet. Suppl. 21, 3-4.

3. Fodor, S. P. A., Read, J. L., Pirrung, M. C., Stryer, L., Lu, A. T. and Solas, D. (1991) Light-directed, spatially addressable parallel chemical synthesis. Science, 251, 767-773.

4. Guschin, D. Y., Mobarry, B. K., Proudnikov, D., Stahl, D. A., Rittmann, B. E. and Mirzabekov, A. D. (1997) Oligonucleotide microchips as genosensors for determinative and environmental studies in microbiology. Appl. Environ. Microbiol., 63, 2397-2402.

5. Rogers, Y. H.; Baucom, P. J.; Huang, Z. J.; Bogdanov, V.; Anderson, S.; Boyce-Jacino, M. T. (1999) Anal. Biochem., 266, 23-30.

6. Guo, Z., Guilfoyle, R. A., Thiel, A. J., Wang, R. and Smith, L. M. (1994) Direct fluorescence analysis of genetic polymorphisms by hybridization with oligonucleotide arrays on glass supports. Nucleic Acid Res. 22, 5456-5465.

7. Patricia, L. D., Wu, Y., Linnea, K. I., Robert, L. M., Mary. A. N., and Gabriel, P. L. (2001) Robust and efficient synthetic method for forming DNA microarrays. Nucleic Acid Res. 29, e107.

8. Lindroos, K.; Liljedahl, U.; Raitio, M.; and Syvänen, A. (2001) Minisequencing on oligonucleotide microarrays: comparison of immobilization chemistries. Nucl. Acids. Res. 29: e69

9. Kumar, A.; Larsson, O.; Parodi, D.; and Liang, Z. (2000) Silanized nucleic acids: a general platform for DNA immobilization Nucl. Acids. Res. 28: e71.

10. Vasiliskov, V. A.; Timofeev, E. N.; Surzhikov, S. A.; Drobyshev, A. L.; Shik, V. V.; Mirzabekov, A. D. (1998) A method for making microchips by copolymerization with acrylamide, Molecular Biology, 32 (5), 780-782.

11. Proudnikov, D., Timofeev, E. and Mirzabekov, A. D., (1998) Immobilization of DNA in polyacrylamide gel for the manufacture of DNA and DNA-oligonucleotide microchips Anal. Biochem., 15934-41.

12. Ermantraut, E., Wohlfart, K., Woelfl, S., Schulz, T. and Koehler, M. (1997) In Ehrfeld, W. (ed), Miroreaction Technology—Proceedings of the First International Conference on Microreaction Technology, Spring Verlag, Heidelberg, pp. 332-339.

13. Benters R, Niemeyer C M, Drutschmann D, Blohm D, Wohrle D, (2002) DNA microarrays with PAMAM dendritic linker systems, Nucl. Acids. Res. 30 (2), U71-77.

14. MacBeath, G.; Schreiber, S. L. (2000) Printing proteins as microarrays for high-throughput function determination, Science 289, 1760-1763.

15. Böhmer, M. R. (1996) Formation and Stability of Multilayers of Polyelectrolytes, Langmuir; 12(15); 3675-3681.

16. Zhou, X. C.; Huang, L. Q.; Li, S. F. Y. (2001) Microgravimetric DNA sensor based on quartz crystal microbalance: comparison of oligonucleotide immobilization methods and the application in genetic diagnosis. Biosensors & Bioelectronics, 16, 85-95.

17. Charlier, V., Laschewsky, A., Meyer, B., Wischerhoff, E.; Multilayers by adsorption of functional polyelectrolytes, MACROMOLECULAR SYMPOSIA, 126, 105-121 January, 1998.

18. Manso-Silvan, A., Fuentes-Cobas, L., Martin-Palma, R. J.; BaTiO3 thin films obtained by sol-gel spin coating, SURF COAT TECH 151, 118-121 Mar. 1, 2002.

We claim:

1. A platform for fabricating a bio-microarray, the platform comprising a solid substrate and a film coating a surface of the solid substrate wherein the film comprises alternating polycationic and polyanionic polymer layers.

2. The platform of claim 1, wherein the solid substrate is made of glass, silica or plastic.

3. The platform of claim 1, wherein the polycationic polymer is selected from polyallylamine hydrochloride (PAAH), polyethyleneimine (PEI), polydimethyldiallylammonium chloride (PDDA), polyacrylamide (PAAM), poly(4-vinylpyridine) hydrochloride (PVP), or an analog of any of the foregoing polymers.

4. The platform of claim 1, wherein the polyanionic polymer is selected from polystyrenesulfonate (PSS), polyvinylsulfonate (PVS), dextrinsulfate (DTS), or an analog of any of the foregoing polymers.

5. The platform of claim 1, wherein the film comprises a pair of a polycationic and polyanionic polymers selected from PAAH/PSS, PAAH/DTS, PEI/PSS, PEI/PVS, PDDA/PSS, PDDA/PVS or PDDA/DTS.

6. The platform of claim 1, wherein the film further comprises metal or NH₄ ions.

7. The platform of claim 6, wherein the metal ions are selected from Mn²⁺, Cu²⁺, Fe²⁺, Na⁺, NH₄⁺, K⁺, Ni²⁺ or Mg²⁺.

8. A bio-microarray comprising the platform of claim 1 and a biological matter attached to the film of the platform.

9. The bio-microarray of claim 8, wherein the biological matter is a biomolecule selected from a polynucleotide or a polypeptide.

10. The bio-microarray of claim 8, wherein the biological matter is selected from a cellular organelle, a cell or a tissue sample.

11. A method for making the platform of claim 1 comprising the steps of:
providing a solid substrate suitable for fabricating bio-microarrays;
coating a surface of the solid substrate with a layer of a first polyionic polymer; and
coating the layer of the first polyionic polymer on the substrate with a layer of a second polyionic polymer wherein the ions of the second polyionic polymer is of the opposite charge to the ions of the first polyionic polymer.

12. The method of claim 11, wherein the coating steps involve exposing the substrate to a solution containing a polyionic polymer.

13. The method of claim 12, wherein the solution is an aqueous solution.

14. The method of claim 12, further comprising the step of adding a salt into the polyionic polymer solution before exposing the solid substrate to the solution.

15. The method of claim 14 wherein the salt is an inorganic salt.

16. A method for fabricating a bio-microarray comprising the steps of:
providing a platform for fabricating a bio-microarray according to claim 1; and
attaching a biological matter to the film of the platform.

17. A platform for fabricating a bio-microarray, the platform comprising a solid substrate and a polyelectrolyte-silica composite film coating a surface of the solid substrate.

18. The platform of claim 17, wherein the polyelectrolyte-silica composite film is formed using a silica sol-gel material that comprises a molecule selected from aminoalkylsiloxanes, aminocarboxyalkylsiloxanes, carboxyalkylsiloxanes or alkoxaysilanes.

19. The platform of claim 17, wherein the solid substrate is made of glass, silica or plastic.

20. The platform of claim 17, wherein the polyelectrolyte is selected from polyallylamine hydrochloride (PAAH), polyethyleneimine (PEI), polydimethyldiallylammonium chloride (PDDA), polyacrylamide (PAAM), poly(4-vinylpyridine) hydrochloride (PVP), polystyrenesulfonate (PSS), polyvinylsulfonate (PVS), dextrinsulfate (DTS), or an analog of any of the foregoing polymers.

21. A bio-microarray comprising the platform of claim 17 and a biological matter attached to the film of the platform.

22. The bio-microarray of claim 21, wherein the biological matter is a biomolecule selected from a polynucleotide or a polypeptide.

23. The bio-microarray of claim 21, wherein the biological matter is selected from a cellular organelle, a cell or a tissue sample.

24. A method for making the platform of claim 17 comprising the steps of:
providing a solid substrate suitable for fabricating bio-microarrays; and
coating a surface of the solid substrate with a polyelectrolyte-silica composite film.

25. The method of claim 24, wherein the coating step involves exposing the substrate to a sol-gel silica solution doped with a polyelectrolyte.

26. The method of claim 24, wherein the polyelectrolyte-silica composite film is coated onto the surface of the solid substrate by the spin-coating method.

27. A method for fabricating a bio-microarray comprising the steps of:
providing a platform for fabricating a bio-microarray according to claim 17; and
attaching a biological matter to the film of the platform.
